(19) United States
(12) Patent Application Publication
(10) Pub. No.: US 2017/0281554 A1
(43) Pub. Date: Oct. 5, 2017

(54) SYSTEM FOR UPPER INTESTINAL DELIVERY OF ACTIVE INGREDIENTS

(71) Applicant: Avion Pharmaceuticals, LLC, Alpharetta, GA (US)

(72) Inventor: Jonathan Bortz, St. Louis, MO (US)

(21) Appl. No.: 15/472,552

(22) Filed: Mar. 29, 2017

Related U.S. Application Data
(60) Provisional application No. 62/314,741, filed on Mar. 29, 2016.

(51) Int. Cl. A61K 9/48; A61K 9/20; A23L 33/16; A61K 33/26

(52) U.S. Cl. CPC A61K 9/4808; A61K 33/26; A61K 9/4825; A61K 9/4816; A61K 9/2086; A23L 33/16; A23V 2002/00

(57) ABSTRACT

A preparation for releasing an active substance, such as iron, in the upper intestine includes an oral dosage form having an external coating, a coating matrix enveloping the oral dosage form, and the active substance embedded in the coating matrix. The active substance disintegrates from the coating matrix within at least one of the stomach, duodenum, upper jejunum, or any combination thereof.

### CLAIM OF PRIORITY

This application is a non-provisional application of U.S. Provisional Application No. 62/314,741, filed Mar. 29, 2016, which is hereby incorporated by reference in its entirety.

### FIELD OF THE INVENTION

The invention relates generally to preparations and methods for releasing and/or delivering an active substance, such as iron, into the upper intestinal gastrointestinal tract and, more specifically, into the upper duodenum.

### BACKGROUND OF THE INVENTION

Gelatin capsules are a widely used dosage form both for pharmaceutical drug products as well as dietary supplements. Gelatin in the presence of certain compounds, mainly aldehydes, or in high humidity and high temperature conditions, can cross-link. Cross-linking involves covalent bonding of the amine group of a lysine side chain of one gelatin molecule to a similar amine group on another molecule. Cross-linking results in the formation of a pellicle on the internal or external surface of the gelatin capsule shell that prevents the capsule fill from being released.

Gelatin is obtained from the partial hydrolysis of collagen, which is the most abundant animal protein in nature. Collagen is an insoluble, highly ordered, fibrous protein. It is the primary fibrous component of bone, skin, and connective tissue. The majority of pharmaceutical gelatin is produced from bovine bone, bovine hide, and porcine skin. Gelatin is graded primarily on the strength of the gel it forms and, depending on the process used and the tissue source, noticeable differences in strength are apparent among suppliers and even between lots from the same supplier. Consequently, controlling the strength of the gel from batch to batch, measured as bloom strength, is key to obtain a consistently performing product. Gelatin manufacturers commonly blend different sublots of gelatin to meet bloom requirements. Bloom strength is a measure of the ability of a given weight of gelatin to set up in water under controlled conditions and is a function of the molecular weight of the gelatin molecules, the concentration of the gelatin in the gel, and the pH of the gel. Bloom strength increases when the gelatin concentration in the gel increases, when the average molecular weight of the gelatin increases, and when the pH of the gel approaches neutrality. In addition, as bloom strength increases, the cost of gelatin increases and gel dissolution rate decreases.

Gelatin capsule shells are prepared from a molten gel mass that is composed of gelatin and a plasticizer dissolved in an aqueous vehicle. For hard gelatin capsules (hardgels) shells, water acts as both the plasticizer and the vehicle. For soft gelatin capsules (softgel) shells, small polyhydroxy compounds such as glycerol, sorbitol, and maltitol typically are used as plasticizers. Although many parameters affect the physical and chemical properties of the shell, the ratio of polymer to plasticizer primarily determines the rigidity, brittleness, and dissolution performance of the shell. Other minor components added to the gel mass may include colorants, flavors, stabilizers, buffers, and opacifiers.

Softgels have a thicker shell and typically exhibit a higher degree of elasticity because of the added plasticizer. They have slightly longer rupture time when compared with hardgels. By comparison, hardgel capsules have a thinner and more rigid shell than do softgel capsules.

The factors that can affect the properties of the gelatin capsule shell include moisture exchange between the shell and the fill material, which can potentially create brittleness in the gelatin shell, and chemical interactions between the fill material and gelatin or between the gelatin and the environment during storage, which can result in gelatin cross-linking. Cross-linking involves strong chemical linkages beyond simple hydrogen and ionic bonding between gelatin chains. One of the strongest and most common types of cross-linking involves the covalent bonding of the amine group of a lysine side chain of one gelatin molecule to a similar amine group on another molecule. This reaction generally is catalyzed by trace amounts of reactive aldehydes. Formaldehyde, glutaraldehyde, glyoxal, and reducing sugars are the most common catalysts. The covalent bonding produced with this type of cross-linking is, for all practical purposes, irreversible, and dissolution of the shell must involve the breaking of other bonds, e.g., by enzyme-mediated breaking of peptide bonds in protein chains. It has been proposed that chemically modified gelatin—by adding succinic acid groups to the lysine side chains—may prevent or at least diminish aldehyde-mediated cross-linking.

The presence of cross-linking will alter the in vitro dissolution behavior of gelatin capsules, resulting in the capsule being unable to open and release its contents into the dissolution medium present in the treatment environment. This failure may not reflect a possible failure to dissolve in the body. The addition of proteolytic enzymes in into the dissolution medium where gelatin capsules or gelatin-coated tablets experience dissolution failure due to the presence of cross-linking may allow dissolution.

However, even when cross-linking does not occur or where it may be so minimal as to not have an impact, the attributes that make gelatin capsules attractive for oral substance delivery create a barrier to the immediate or near-immediate release of their active ingredient payload.

Cross-linking results in the formation of a pellicle on the internal or external surface of the gelatin capsule shell. A pellicle is a thin, water insoluble clear membrane of cross-linked protein on the inner or outer surface of the capsule that prevents the capsule fill from being released. Cross-linking is evidenced by the observation of a thin membrane or gelatinous mass during dissolution testing because the pellicle itself may be difficult to observe.

Another, weaker, type of cross-linking is complexation of free carboxylic acid groups on two different gelatin molecules with trivalent metal ions, such as Fe³⁺ and Al³⁺. These cations may be found in some of the dyes used as colorants or as low levels of contaminants in excipients. Higher bloom gelatin, which is normally associated with higher quality, facilitates efficient cross-linking because fewer links are needed to join greater lengths of gelatin chains.

The active substances, such as nutrients, minerals, vitamins, pharmaceutical ingredients, alone or in combination, that are encapsulated in gelatin capsules must be released in certain time frames in order to be effective for their purposes. One such time frame for the release of a nutritional substance is the release of iron in the gastrointestinal tract. Typically, iron is only absorbed into the body when the pH of the absorbing environment is less than about 3. In such a pH range, iron may be readily absorbed into the body, but in higher pH ranges, iron is either more slowly, or not at all, absorbed into the body. When iron and other nutrients are encapsulated by polymer coatings forming gelatin capsule, their release is typically delayed and there is less total absorption by the body.

The absorption of iron in the form of ferrous and ferric iron salts occurs in the proximal duodenum where these two iron forms are most likely to be in solution. The degree of solubility is closely tied to the pH of duodenal fluid and because the pH is below 3 only up until the mid-point of the duodenum where the pancreatic duct deposits enough sodium bicarbonate to raise the pH above 3, even ferrous iron crystallizes and can no longer be absorbed by the duodenal enterocyte. Even chelated iron, while not being as tied to pH for bioavailability and having been described as being able to be absorbed more distally, it is likely that even the site of this absorption is in the upper small intestine. This is significant when iron is the only ingredient in or is only part of a multi-ingredient payload, in which the release of iron into the gastric, duodenal or jejunal fluid is impaired or prevented completely by failure of the gelatin soft capsule to disintegrate in a way that allows any of the contents to escape.

Therefore, there at least remains a need in the art for preparations and nutritional supplements that permit the release of active substances (e.g., drugs, nutrients such as iron, and the like) in the body while avoiding the negative effects of cross-linking of the gelatin capsule coating.

### SUMMARY OF THE INVENTION

One or more example embodiments may address one or more of the aforementioned problems. Certain example embodiments provide a preparation, such as a pharmaceutical preparation or nutritional supplement, in the form of a gelatin capsule designed to release an active substance, such as iron, into the upper intestine, and more specifically, the stomach, the duodenum, the upper duodenum, or the upper jejunum. In accordance with certain embodiments, the preparation may comprise an oral dosage form having an external coating, a coating matrix enveloping the oral dosage form, and the active substance embedded partially or wholly in the coating matrix. In such embodiments, the active substance may disintegrate from the coating matrix within at the upper intestinal region, which could comprise at least one of the stomach, duodenum, upper jejunum, or any combination thereof.

In another aspect, a method of delivering an active substance to an upper intestinal region of a subject is provided. The method may comprise forming a preparation and administering the preparation to the subject. In such embodiments, the preparation may comprise an oral dosage form having an external coating, a coating matrix enveloping the oral dosage form, and an active substance, such as iron, embedded in the coating matrix such that the active substance disintegrates from the coating matrix within the upper intestinal region of the subject.

### DETAILED DESCRIPTION OF EXEMPLARY EMBODIMENTS

Some example embodiments now will be described more fully hereinafter. Indeed, the examples described herein should not be construed as being limiting as to the scope, applicability, or configuration of the present disclosure. Rather, these example embodiments are provided so that this disclosure will satisfy applicable legal requirements. As used in the specification, and in the appended claims, the singular forms “a”, “an”, “the”, include plural referents unless the context clearly dictates otherwise.

Certain example embodiments provide preparations, including but not limited to preparation and nutritional supplements, and methods for releasing an active substance, such as iron in various forms, in the upper intestine. For instance, such preparations and methods may provide, for example, an effective manner for efficiently releasing active substances in the body while avoiding the negative effects of cross-linking. As such, for example, these preparations and methods may permit faster release and increased bioavailability of active ingredients in the body.

In some example embodiments, a preparation for releasing an active substance in the upper intestine is provided. For instance, the preparation provides an effective manner for efficiently releasing active substances in the body while avoiding the negative effects of cross-linking. As such, for example, these preparations and methods may permit faster release and increased bioavailability of active ingredients in the body. In one aspect, the preparation for releasing an active substance in the upper intestine may include an oral dosage form having an external coating, a coating matrix enveloping the oral dosage form, and the active substance embedded in the coating matrix. In such embodiments, for example, the active substance may disintegrate from the coating matrix within at least one of the stomach, duodenum, upper jejunum, or any combination thereof. According to certain exemplary embodiments, for instance, the preparation may further comprise at least one of a fish oil additive, a calcium additive, or any combination thereof.

The invention exemplified herein contemplates piggy-backing the active ingredient (by way of example, iron) in an external coating that will form a matrix with a polymer selected to be soluble in a pH of less than about 5.0 and, in some cases, less than about 3.0 such that the active ingredient may be absorbed into the human body in the upper intestinal tract when a preparation containing the active ingredient is administered.

In accordance with certain exemplary embodiments, for example, the oral dosage form may comprise a soft gel capsule, a hard shell gel capsule, a caplet, or a tablet. In some embodiments, for instance, the coating matrix may comprise a matrix formed between the external coating and a polymer soluble in a pH less than 5.0 (e.g., less than 4.5, less than 4.0, less than 3.5, less than 3.0, less than 2.5, less than 2.0, and less than 1.5. In one embodiment, the polymer will be soluble in a pH of less than about 5.0 and in another embodiment, the polymer will be soluble in a pH of less than about 3.0.

In accordance with certain exemplary embodiments, for example, the active substance may comprise at least one of a nutrient, a mineral, a vitamin, a pharmaceutical ingredient, or any combination thereof. In certain embodiments, for instance, the active substance may comprise a mineral. In some embodiments, for example, the mineral may comprise at least one of an iron, a calcium, a magnesium, a zinc, an iodine, or any combination thereof. In other embodiments, for instance, the mineral may comprise an iron. In further embodiments, for example, the iron may comprise at least one of a ferrous salt, a ferrous complex, a ferrous chelate, a ferric salt, a ferric complex, a ferric chelate, or any combination thereof. In some embodiments, for instance, the iron may comprise a ferrous chelate. In such embodiments, for example, the ferrous chelate comprises ferrous bisglycinate, ferrous asparto-glycinate, or any combination thereof.

In accordance with certain exemplary embodiments, for instance, disintegration of the active substance from the coating matrix may begin from about 1 minute to about 15 minutes after ingestion. According to certain exemplary embodiments, for example, disintegration of the active substance from the coating matrix may be from about 40% to about 100% complete 60 minutes after ingestion. In some embodiments, for instance, disintegration of the active substance from the coating matrix may be from about 60% to about 100% complete 60 minutes after ingestion. In further embodiments, for example, disintegration of the active substance from the coating matrix may be from about 80% to about 100% complete 60 minutes after ingestion. As such, in certain embodiments, disintegration of the active substance from the coating matrix may be at a percentage 60 minutes after ingestion from at least about any of the following: 40, 45, 50, 55, 60, 65, 70, 75, and 80% and/or at most about 100, 95, 90, 85, and 80% (e.g., about 50-100%, about 70-95%, etc.).

In accordance with certain exemplary embodiments, for instance, the coating matrix may comprise an active substance content. In such embodiments, for example, dissolution of the active substance may comprise from about 20% to about 100% of the active substance content within 30 minutes after ingestion. As such, in certain embodiments, dissolution of the active substance content within 30 minutes after ingestion may comprise from at least about any of the following: 20, 25, 30, 35, 40, 45, and 50% and/or at most about 100, 95, 90, 85, 80, 75, 70, 65, 60, and 55% (e.g., about 20-85%, 30-100%, etc.).

In certain embodiments, for instance, dissolution of the active substance may comprise from about 40% to about 100% of the active substance content within 60 minutes after ingestion. In other embodiments, for example, dissolution of the active substance may comprise from about 60% to about 100% of the active substance content within 60 minutes after ingestion. In further embodiments, for instance, dissolution of the active substance may comprise from about 80% to about 100% of the active substance content within 60 minutes after ingestion. As such, in certain embodiments, dissolution of the active substance content within 60 minutes after ingestion may comprise from at least about any of the following: 40, 45, 50, 55, 60, 65, 70, 75, and 80% and/or at most about 100, 95, 90, 85, and 80% (e.g., about 45-90%, about 65-100%, etc.).

In another aspect, certain exemplary embodiments provide a method of delivering an active substance to an upper intestinal region of a subject. For instance, this method provides an effective manner for efficiently releasing active substances in the body while avoiding the negative effects of cross-linking. As such, for example, these preparations and methods may permit faster release and increased bioavailability of active ingredients in the body. According to certain embodiments, for example, the method may include forming a preparation, and administering the preparation to the subject. In such embodiments, for instance, the preparation may comprise an oral dosage form having an external coating, a coating matrix enveloping the oral dosage form, and an active substance embedded in the coating matrix such that the active substance disintegrates from the coating matrix within the upper intestinal region of the subject. According to certain exemplary embodiments, for example, the preparation may further comprise at least one of a fish oil additive, a calcium additive, or any combination thereof.

In accordance with certain exemplary embodiments, for example, the oral dosage form may comprise a soft gel capsule, a hard shell gel capsule, a caplet, or a tablet in the forms and with the active ingredients, coatings and polymers described herein. The oral dosage form may be made according to known techniques for creating various pharmaceutical preparations and nutritional supplements. Such techniques may include, but are not limited to, the formation of capsules such as gelcaps by spraying and dipping. In any such processes, the active substances set forth in the present disclosures are embedded in the coatings and are applied to the remaining preparation payload by including the active substances in the coating layers that are being sprayed onto or dipped around the remaining preparation payload or by other techniques that reach the same result.

One form polymer found to be soluble at a pH of less than 5.0 and useful in the present invention is Eudragit® E PO ReadyMix available from Evonik Industries of Darmstadt, Germany. Eudragit® E PO ReadyMix is a powder blend polymer that may be used as the gelatin capsule external coating of the present invention. The coating can easily be prepared by only adding water and subjecting the mix to either approximately 30 minutes of high-shear mixing or approximately 60 minutes of propeller mixing. Other components, in addition to the active ingredient (such, by example, iron) may be utilized: sodium lauryl sulfate, talc, silicon dioxide, and stearic acid, to name a few exemplary components. Optional ingredients such as color pigments (e.g., iron oxides, aluminum flakes, and natural colors), flavors and fragrances, and others may also be employed to produce the preparation.

The active ingredient may be added in various proportions (including up to a full effective load) into the polymer coating.

Once formed, the coating with embedded active ingredient can be applied by a typical spraying process to form the gelatin capsule. Typically, this will be a low-temperature process. The resulting gelatin capsule will be readily absorbable when it reaches the upper intestinal tract and will release its embedded active ingredient.

The resulting release of iron, or other active, occurs immediately upon contact with the low pH of the stomach. Therefore, the active ingredient becomes capable of being absorbed in the duodenum or upper jejunum.

Other ingredients that are absorbed distally in jejunum or ileum are much less affected by delays in disintegration and dissolution. There is an additional benefit in multi-ingredient formulations that contain fish oil together other solid ingredients like calcium that typically turn the content of the gelcap into a thick paste from which iron may have greater difficulty being dissolved in upper gastro-intestinal solution because of the amount of time it takes to diffuse out of the paste-like content. Even when a gelcap is sliced open and placed in an in-vitro simulated gastric and intestinal fluid environment, iron may not be found dissolved in solution in a dissolution test performed on both an intact gelcap containing multiple ingredients of a prenatal formulation, even if the gelcap is sliced in three places and held into gastric solution by a sinker device.

Many modifications and other embodiments will come to mind to one skilled in the art to which this disclosure pertains having the benefit of the teachings presented in the foregoing descriptions. Therefore, it is to be understood that this disclosure is not to be limited to the specific embodiments disclosed and that modifications and other embodiments are intended to be included within the scope of the appended claims. Moreover, although the foregoing descriptions describe exemplary embodiments in the context of certain exemplary combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. In cases where advantages, benefits or solutions to problems are described herein, it should be appreciated that such advantages, benefits and/or solutions may be applicable to some example embodiments, but not necessarily all example embodiments. Thus, any advantages, benefits or solutions described herein should not be thought of as being critical, required or essential to all embodiments or to that which is claimed herein. Although specific terms are employed herein, they are used in a generic and descriptive sense only and not for purposes of limitation.

What is claimed is:

1. A preparation for releasing iron in the upper intestine, the preparation comprising:
an oral dosage form having an external coating;
a coating matrix enveloping the oral dosage form; and
iron embedded in the coating matrix,
wherein the active substance disintegrates from the coating matrix within at least one of the stomach, duodenum, upper jejunum, or any combination thereof.

2. The preparation of claim 1 wherein the coating matrix contains all of the iron present in the preparation.

3. The preparation of claim 1 wherein the coating matrix contains only a portion of the iron present in the preparation.

4. The preparation of claim 1, wherein the oral dosage form comprises a soft gel capsule, a hard shell gel capsule, a caplet, or a tablet.

5. The preparation of claim 1, wherein the coating matrix comprises a matrix formed between the external coating and a polymer soluble in a pH less than about 5.0.

6. The preparation of claim 1, wherein the coating matrix comprises a matrix formed between the external coating and a polymer soluble in a pH less than about 3.0.

7. The preparation of claim 1, wherein the iron comprises at least one of a ferrous salt, a ferrous complex, a ferrous chelate, a ferric salt, a ferric complex, a ferric chelate, or any combination thereof.

8. The preparation of claim 7, wherein the ferrous chelate comprises ferrous bisglycinate, ferrous asparto-glycinate, or any combination thereof.

9. The preparation of claim 1, wherein disintegration of the iron from the coating matrix begins from about 1 minute to about 15 minutes after ingestion.

10. The preparation of claim 1, wherein disintegration of the iron from the coating matrix is from about 40% to about 100% complete 60 minutes after ingestion.

11. The preparation of claim 1, wherein disintegration of the iron from the coating matrix is from about 60% to about 100% complete 60 minutes after ingestion.

12. The preparation of claim 1, wherein disintegration of the iron from the coating matrix is from about 80% to about 100% complete 60 minutes after ingestion.

13. The preparation of claim 1, wherein the coating matrix comprises an iron content, and dissolution of the iron comprises from about 20% to about 100% of the iron content within 30 minutes after ingestion.

14. The preparation of claim 1, wherein dissolution of the iron comprises from about 40% to about 100% of the iron content within 60 minutes after ingestion.

15. The preparation of claim 1, wherein dissolution of the iron comprises from about 60% to about 100% of the iron content within 60 minutes after ingestion.

16. The preparation of claim 1, wherein dissolution of the iron comprises from about 80% to about 100% of the iron content within 60 minutes after ingestion.

17. The preparation of claim 1, further comprising at least one of a fish oil additive, a calcium additive, sodium lauryl sulfate, talc, silicon dioxide, and stearic acid, or any combination thereof.

18. A method of delivering iron to an upper intestinal region of a subject, the method comprising:
forming a preparation, wherein the preparation comprises:
an oral dosage form having an external coating,
a coating matrix enveloping the oral dosage form, and
iron embedded in the coating matrix; and
administering the preparation to the subject,
wherein the iron disintegrates from the coating matrix within the upper intestinal region of the subject.

19. The method of claim 17, wherein the oral dosage form comprises a soft gel capsule, a hard shell gel capsule, a caplet, or a tablet.

20. The method of claim 17, wherein the coating matrix comprises a matrix formed between the external coating and a polymer soluble in a pH less than about 5.0.

21. The method of claim 17, wherein the coating matrix comprises a matrix formed between the external coating and a polymer soluble in a pH less than about 3.0.
